(19) United States
(12) Patent Application Publication
(10) Pub. No.: US 2014/0268040 A1
(43) Pub. Date: Sep. 18, 2014

(54) Multimodal Ocular Imager

(71) Applicant: Physical Sciences, Inc., (US)

(72) Inventors: Mircea Mujat, Acton, MA (US); R. Daniel Ferguson, Melrose, MA (US); Nicusor Iftimia, Chelmsford, MA (US); David Biss, Brighton, MA (US)

(21) Appl. No.: 13/827,905

(22) Filed: Mar. 14, 2013

(51) Int. Cl. A61B 3/10

(52) U.S. Cl. CPC A61B 3/102
USPC 351/206; 351/246

(57) ABSTRACT

An imaging probe for imaging a sample includes at least one light source that provides light to a sample. The imaging probe also includes a first imaging objective that focuses a first light reflected from the sample and a second light reflected from the sample, such that the first light and the second light share at least a portion of an imaging path. The imaging probe also includes a first optical component that receives the first light and the second light from the first imaging objective, directs the first light towards an imaging camera to obtain a first image of the sample, and directs the second light toward an optical coherence tomography (OCT) imaging apparatus to obtain a second image of the sample.

### GOVERNMENT RIGHTS

The invention was made with government support under U.S. Army Medical Research and Materiel Command contract number W81XWH-12-C-0116. The government may have certain rights in the invention.

### FIELD OF THE INVENTION

This invention relates, generally, to optical imaging, and more particularly, to apparatuses and methods for imaging anterior and posterior segments of a sample (e.g., eye) with optical coherence tomography and an imaging camera.

### BACKGROUND OF THE INVENTION

Images of a sample (e.g., eye) can be obtained with optical coherence tomography (OCT) and/or digital cameras. OCT images and digital camera images are typically obtained with separate instruments (e.g., imaging devices).

Digital camera devices (e.g., charge-coupled device cameras) can be used in both industrial and medical applications, for example, to obtain two-dimensional images of a sample. Instruments employing digital camera devices can be used in ophthalmology for examining and imaging anterior segments of an eye including, for example, a cornea, sclera, iris, and/or lens. Instruments employing digital camera devices (e.g., fundus or retinal cameras) can also be used to create a fundus photograph, e.g., a photograph of a posterior segment of an eye including the retina and/or other structures of the eye.

Optical coherence tomography (OCT) can be used in both industrial and medical applications to obtain three-dimensional images of a sample. For example, OCT instruments can be used in ophthalmology for imaging areas of anterior (e.g., cornea and sclera) and posterior (e.g., retina) segments of an eye. Current systems can profile the eye anterior segment (e.g., using laser profilometry and/or ultrasound imaging), however these systems typically cannot be converted for retinal imaging of the eye and/or they produce images with resolutions that are insufficient for ophthalmologic applications (e.g., resolutions approximately greater than 100 μm).

When using OCT to obtain an image of an anterior segment of an eye, the image of the cornea and sclera can be limited by a palpebral aperture of the eye. Applications that can utilize an OCT image of an anterior segment of an eye can include fitting contact lenses and/or fitting scleral lenses (e.g., Prosthetic Replacement for the Ocular Surface Environment [PROSE] devices developed by Boston Foundation for Sight).

Scleral lenses are hard contact lenses that can cover a cornea of an eye and/or a large portion of a sclera of the eye to provide, for example, an environment for a corneal surface of the eye to heal following injury to the eye. Scleral lenses can have wide applicability in treating, for example, military personnel who have suffered third degree facial burns from explosions or chemical exposure. Furthermore, scleral lenses can be used to treat corneal diseases including ectasia, keratoconjunctivitis sicca, and extreme cases of keratoconus.

OCT imaging can be used to create an image of an eye's surface topography to aid contact lens manufacturers in producing scleral lenses that are tailored to a patient's particular eye condition. One problem with current OCT imaging instruments is that they can have a limited practical scan size and typically cannot perform topography measurements over large areas, for example, areas including portions of the sclera adjacent to the cornea. Current methods to increase the size of the OCT image include further opening the eyelid with a speculum, or similar device. This can be uncomfortable for the patient, increase the duration of an office visit, and/or result in eyelid or eye injuries to the patient.

Current methods for fitting scleral lenses can be inexact and can include inexact empirical processes. Current methods for fitting scleral lenses can often require fitting a patient with several contact lenses, each having slightly different shapes, over multiple clinical visits. The fitting sessions can be painful to the patient.

Completing a comprehensive eye examination for disease diagnosis can require an ophthalmologist to subject a patient to several imaging procedures performed with different instruments (e.g., four different instruments). Each instrument can be specialized for imaging either the anterior or the posterior segment of the eye. The instruments can use photography or OCT. An imaging procedure with each instrument can require a patient to be positioned in a different seating position and/or head stabilizer. Individual instruments can be too large to fit within the same examination room. An imaging procedure with multiple instruments can require a patient to be examined in several different examination rooms.

Therefore, it is desirable to obtain retinal imaging and fundus photography of a sample (e.g., a posterior segment of an eye) with a single instrument. It is also desirable to obtain topographical and/or two-dimensional imaging of a sample (e.g., an anterior segment of an eye) with a single instrument. It is also desirable to perform multiple imaging procedures with a patient positioned a single seating position and/or head stabilizer. It is also desirable to use less office space for imaging instruments. It is also desirable to perform up to four different imaging procedures in a single examination room. It is also desirable to reduce the cost of imaging instrumentation.

It is also desirable to obtain an image of an eye that displays more of a cornea and/or sclera than is typically visible via a palpebral aperture when taking an OCT image for, e.g., fitting contact lenses and/or scleral lenses.

It is also desirable to limit the duration of contact lens and/or scleral lens fittings. It is also desirable to limit patient discomfort. It is also desirable to reduce the cost of treatment by, for example, reducing the number of office visits required by a patient for a fitting. It is also desirable to increase the number of patients that can be treated on a daily basis by, for example, limiting the duration of an office visit. It is also desirable to obtain images of portions of the sclera adjacent to a cornea of an eye. It is also desirable for an imaging instrument that accounts for involuntary movements of an eye that can occur during imaging.

### SUMMARY OF THE INVENTION

One advantage of the invention includes obtaining retinal imaging and fundus photography of a sample (e.g., a posterior segment of an eye), with a single instrument. Another advantage of the invention includes obtaining topographical and/or two-dimensional imaging of a sample, (e.g., an anterior segment of an eye), with a single instrument. Another advantage of the invention includes performing multiple imaging procedures with a patient positioned in a single seating position and/or head stabilizer. Another advantage of the invention includes using less office space for imaging instruments. Another advantage of the invention includes performing four different imaging procedures in a single examination room. Another advantage of the invention includes reducing the cost of imaging instrumentation.

Another advantage of the invention includes obtaining an image of an eye that displays more of the cornea and sclera than is typically visible via the palpebral aperture when taking an OCT image for, e.g., fitting contact lenses or scleral lenses.

Another advantage of the invention includes limiting the duration of contact lens and/or scleral lens fittings. Another advantage of the invention includes limiting patient discomfort. Another advantage of the invention includes reducing the cost of treatment by, for example, reducing the number of office visits required by a patient for a fitting. Another advantage of the invention includes increasing the number of patients that can be treated on a daily basis by, for example, limiting the duration of an office visit. Another advantage of the invention includes obtaining images of portions of the sclera adjacent to a cornea of an eye. Another advantage of the invention includes an imaging instrument that accounts for involuntary movements of an eye that can occur during imaging.

In one aspect, the invention includes an imaging probe for imaging a sample. The imaging probe includes at least one light source that provides light to a sample. The imaging probe also includes a first imaging objective that focuses a first light reflected from the sample and a second light reflected from the sample, such that the first light and the second light share at least a portion of an imaging path. The imaging probe also includes a first optical component that receives the first light and the second light from the first imaging objective, directs the first light towards an imaging camera to obtain a first image of the sample, and directs the second light toward an optical coherence tomography (OCT) imaging apparatus to obtain a second image of the sample.

In some embodiments, the first optical component directs a first beam of light received from the OCT imaging apparatus toward the first imaging objective which focuses the first bean of light toward the sample.

In some embodiments, the imaging probe includes at least one light source that is a light-emitting diode (LED).

In some embodiments, the first optical component includes a dichroic beam splitter. In some embodiments, the first optical component substantially reflects infrared light, and substantially transmits visible light.

In some embodiments, the imaging probe includes a second imaging objective positioned between the imaging camera and the first optical component that focuses the first beam of light toward the imaging camera.

In some embodiments, the imaging probe includes a collimator positioned between the OCT imaging apparatus and the first optical component that collimates the third beam of light.

In some embodiments, the sample is an eye, the first image is a two-dimensional image of an anterior surface of the eye, and the second image is an OCT image of the anterior surface of the eye.

In some embodiments, the sample is an eye, and an ophthalmic lens is positioned between the first imaging objective and the sample such that the first image is a fundus photograph of the eye, and the second image is an OCT image of the retina of the eye.

In another aspect, the invention involves a method for imaging a sample. The method involves illuminating a sample with a first beam of light from a light-emitting diode (LED) and a second beam of light from an optical coherence tomography (OCT) imaging apparatus. The method also involves focusing a first light reflected from the sample towards an imaging camera to obtain a first image of the sample, and focusing a second light reflected from the sample towards the OCT imaging apparatus to obtain a second image of the sample, such that the first light and the second light share at least a portion of an imaging path.

In some embodiments, illuminating the sample with a second beam of light also involves directing the second beam of light toward a first imaging objective.

In some embodiments, the method also involves illuminating the sample at a first set of predetermined positions. In some embodiments, the method also involves obtaining a third image of the sample and a fourth image of the sample while the sample is illuminated at the first set of predetermined positions, wherein the third image of the sample is a two-dimensional image, and the fourth image of the sample is an OCT image.

In some embodiments, the method also involves determining a set of rotation reference positions based on the first image of the sample and the third image of the sample. In some embodiments, the method also involves determining a combined image of the sample based on the second image of the sample, the set of rotation reference positions, and the fourth image of the sample, such that the combined image of the sample shows an area of the sample that is larger than the second image of the sample and the fourth image of the sample.

In some embodiments, focusing a first light involves focusing visible light’ and focusing a second beam of light further comprises focusing infrared light.

In some embodiments, focusing a first light also involves receiving the first light by a second imaging objective, and focusing the first light toward the imaging camera.

In some embodiments, illuminating a sample with a second beam of light also involves collimating the second beam of light.

In some embodiments, the sample is an eye, the first image is a two-dimensional image of the anterior surface of the eye, and the second image is an OCT image of the anterior surface of the eye.

In some embodiments, the sample is an eye. In some embodiments, the method also involves positioning an ophthalmic lens between the first imaging objective and the eye such that the first image is a fundus photograph of the eye, and the second image is an OCT image of the retina of the eye.

In some embodiments, the first set of predetermined positions is based on an amount of desired overlap between the second image of the sample and the fourth image of the sample. In some embodiments, the desired overlap is half of the second image of the sample overlapping with half of the fourth image of the sample.

In some embodiments, determining a combined image of the sample also involves determining whether a height difference between the second image of the sample and the fourth image of the sample is within a desired threshold.

In some embodiments, determining a quality value of the second image of the sample is based on an amount the sample moves during imaging.

In another aspect, the invention involves a method for obtaining an image of an eye. The method involves obtaining a first optical coherence tomography (OCT) image of the eye. The method also involves obtaining a first two-dimensional image of the eye. The method also involves illuminating the eye at a first set of predetermined positions. The method also involves obtaining a second OCT image of the eye and a second two-dimensional image of the eye while the eye is illuminated at the first set of predetermined positions. The method also involves determining a set of rotation reference positions based on the first two-dimensional image of the eye and the second two-dimensional image of the eye. The method also involves determining a combined image of the eye based on the first OCT image of the eye, the set of rotation reference positions, and the second OCT image of the eye, such that the combined image of the eye shows an area of the eye that is larger than the first OCT image of the eye and the second OCT image of the eye.

In some embodiments, the method also involves illuminating the eye at a second set of predetermined positions, wherein the first set of predetermined positions and the second set of predetermined positions are different. In some embodiments, the method also involves obtaining a third OCT image of the eye and a third two-dimensional image of the eye while the eye is illuminated at the second set of predetermined positions.

In some embodiments, the method also involves determining a second set of rotation reference positions based on the first two-dimensional image of the eye and the third two-dimensional image of the eye. In some embodiments, the method also involves determining a second combined image of the eye based on the second set of rotation reference positions, the combined image, and the third OCT image of the eye, such that the second combined image shows an area of the eye that is larger than the combined image and the third OCT image of the eye.

In some embodiments, the first set of predetermined positions is based on an amount of desired overlap between the first OCT image of the eye and the second OCT image of the eye. In some embodiments, the desired overlap is half of the first OCT image of the eye overlapping with half of the second OCT image of the eye.

In some embodiments, determining a combined image of the eye also involves determining whether a height difference between the first OCT image of the eye and the second OCT image of the eye is within a desired threshold.

In some embodiments, the method also involves determining the quality of the first OCT image of the eye with the second OCT image of the eye based on a comparison of the height difference between the first OCT image of the eye and the second OCT image of the eye with a predetermined threshold.

In some embodiments, the method also involves determining a quality value of the first OCT image of the eye based on an amount the sample moves during imaging. In some embodiments, the method also involves determining a quality value of the second OCT image of the eye based on an amount the sample moves during imaging.

### BRIEF DESCRIPTION OF THE DRAWINGS

The advantages of the invention described above, together with further advantages, may be better understood by referring to the following description taken in conjunction with the accompanying drawings. The drawings are not necessarily to scale; emphasis instead is generally placed upon illustrating the principles of the invention.

FIG. 1 is a diagram of an imaging apparatus, according to an illustrative embodiment of the invention.

FIG. 2 is a flow chart illustrating a method of imaging a sample, according to an embodiment of the invention.

FIG. 3 is a flow chart illustrating a method for obtaining in image of an eye, according to an illustrative embodiment of the invention.

FIG. 4A is a diagram of an exemplary eye, according to an illustrative embodiment of the invention.

FIG. 4B shows plots of images of the eye of FIG. 4A, according to illustrative embodiments of the invention.

FIG. 5 is a diagram of an eye, according to an illustrative embodiment of the invention.

FIG. 6 shows exemplary images of a moving eye, according to an illustrative embodiment of the invention.

FIGS. 7A-7B show graphs of images of an anterior segment of an eye, according to an illustrative embodiment of the invention.

FIG. 8 shows a graph of a mean height difference between multiple OCT images, according to an illustrative embodiment of the invention.

### DETAILED DESCRIPTION OF THE INVENTION

FIG. 1 is a diagram of an imaging apparatus 100 in accordance with an illustrative embodiment of the invention. The imaging apparatus 100 includes an imaging probe 101, an OCT imaging apparatus 102, a light source 103, a system control and data processing unit 104, light-emitting diode (LED) 115a, LED 115b, generally LEDs 115, a first imaging objective 114, and a third imaging objective 116.

The imaging probe 101 is in communication with the first imaging objective 114, LEDs 115, the system control and data processing unit 104, and the OCT imaging apparatus 102. The OCT imaging apparatus 102 is in communication with the system control and data processing unit 104 and the light source 103. The first imaging objective 114 includes two lenses, a first lens 114a, and a second lens 114b.

The imaging probe 101 includes a waveguide 117, a collimator 105, galvanometers 106 and 108, a first optical component 107, a second optical component 109, a reflective optical component 111, a second imaging objective 112, and an imaging camera 113.

The waveguide 117 is in communication with the OCT imaging apparatus 102 and the collimator 105. The collimator 105 is in communication with the first optical component 107. The first optical component 107 is in communication with the galvanometer 106 and the second optical component 109. Galvanometer 106 is in communication with the system control and data processing unit 104.

The second optical component 109 is in communication with the galvanometer 108, the reflective optical component 111, and the first imaging objective 114. Galvanometer 108 is in communication with the system control and data processing unit 104. The reflective optical component 111 is in communication with the second imaging objective 112. The second imaging objective 112 is in communication with the imaging camera 113. The imaging camera 113 is in communication with the system control and data processing unit 104.

The first imaging objective 114 is in communication with the second optical component 109 and the third imaging objective 116. The third imaging objective 116 can be an ophthalmic lens (e.g., a Volk lens). In some embodiments, the third imaging objective 116 is not present, and the first imaging objective 114 is in communication with the second optical component 109 and the sample 110. In some embodiments, the second optical component 109 is not present, and the first imaging objective 114 is in communication with the first optical component 107.

The third imaging objective 116 is in communication with the first imaging objective 114 and the sample 110. The LEDs 115 are in communication with the sample 110 and the imaging probe 101. In some embodiments, the imaging LEDs 115 are in communication with the system control and data processing unit 104.

In some embodiments, the first imaging objective 114 includes only one lens. In some embodiments, the first imaging objective 114 includes three or more lenses. In some embodiments, the first imaging objective 114 is a compound lens. In some embodiments, the first imaging objective 114 is a telecentric lens. In some embodiments, the first imaging objective 114 comprises achromatic lenses.

In some embodiments, the imaging apparatus 100 has four LEDs. In some embodiments, the imaging apparatus 100 includes any number of LEDs. In some embodiments, the LEDs 115 are housed within in the imaging probe 101. In some embodiments, the LEDs are controlled (e.g., turned on, turned off, and/or turned partially on) by the imaging apparatus 101.

In some embodiments, the LEDs are controlled by the system control and data processing unit 104. In some embodiments, the LEDs are pre-programmed to turn on, turn off, and/or turn partially on.

In some embodiments, the first optical component 107 is a scanning mirror. In some embodiments, the first optical component 107 has a surface that substantially reflects infrared light. In some embodiments, the second optical component 109 is a dichroic beam splitter. In some embodiments, the second optical component 109 has a surface that substantially reflects infrared light, and substantially transmits visible light.

In some embodiments, the reflective optical component 111 is a mirror. In some embodiments, the imaging probe 101 does not include the reflective optical component 111, and the second imaging objective 112 is positioned such that it can receive light directly from the second optical component 109.

In some embodiments, the imaging camera 113 is any camera capable of obtaining a two-dimensional image of the sample. In some embodiments, the imaging camera 113 includes a charge-coupled device (CCD) image sensor. In some embodiments, the imaging camera 113 includes a complementary metal-oxide-semiconductor (CMOS) image sensor.

In some embodiments, the imaging probe 101 does not include the first optical component 107, the second optical component 109, and galvanometers 106 and 108. In these embodiments, the imaging probe can include a single, multi-axis galvanometer and a single optical element capable of directing the first light in two different dimensions (e.g., the dimensions that the first optical component 107 and the second optical component 109 direct the first light).

It is understood by one of ordinary skill in the art that the components described in FIG. 1 can be housed in any variety of configurations. For example, in some embodiments, the imaging probe 101, the OCT imaging apparatus 102, the light source 103, the first imaging objective 114, and the LEDs 115 are housed in a single housing. In some embodiments, imaging probe 101, the OCT imaging apparatus 102, the light source 103, the first imaging probe 114, and the LEDs 115 are each in their own housing. In some embodiments, the OCT imaging apparatus 102 and light source 103 are housed in a single housing.

In some embodiments, the OCT imaging apparatus 102 is a time-domain OCT imaging apparatus, a Fourier domain OCT imaging apparatus, a swept source OCT imaging apparatus, or any OCT imaging apparatus that is known in the art. For example, see the OCT imaging apparatus as described in Optical Coherence Tomography—Principles and Applications by Fercher et al., Rep. Prog. Phys. Vol. 66, No. 2 (2003), pp. 239-303.

In some embodiments, the system control and data processing unit 104 includes a quad-core central processing unit (CPU), a frame grabber, a real-time graphical processing unit (GPU), an analog output data acquisition (DAQ) card, and/or controllers for the OCT scanning devices.

During operation, a first beam of light emits from the light source 103 and impinges upon the OCT imaging apparatus 102. A waveguide 117 guides the first beam of light from the OCT imaging apparatus 102 to the collimator 105 of the imaging probe 101. The collimator 105 collimates the first beam of light. The collimated first beam of light impinges upon the first optical component 107.

Galvanometer 106 positions the first optical component 107 to deflect the first beam of light in a first dimension (e.g., vertical or Y-direction) of the sample 110 to be imaged. The galvanometer 106 can receive a control signal that indicates the position for the first optical component 107. The control signal can be provided by the system control and data processing unit 104. In some embodiments, the control signal is programmed into the galvanometer 106 and/or the imaging probe 101. The control signal can depend on a size (e.g., area) of the sample to be imaged in the first dimension, a desired imaging speed, and/or a geometric shape of an image scanning pattern.

The deflected first beam of light impinges upon the second optical component 109. Galvanometer 108 positions the second optical component 109 to deflect the first beam of light in a second dimension (e.g., horizontal or X-direction) of the sample 110 to be imaged. The galvanometer 108 can receive a control signal that indicates the position for the second optical component 109. The control signal can be provided by the system control and data processing unit 104. In some embodiments, the control signal is programmed into the galvanometer 108 and/or the imaging probe 101. The control signal can depend on a size (e.g., area) of the sample to be imaged in the second dimension, a desired imaging speed, and/or a geometric shape of an image scanning pattern.

The deflected first beam of light impinges upon the first imaging objective 114. The first imaging objective 114 focuses the first beam of light towards the third imaging objective 116. The third imaging objective 116 focuses the first beam of light towards the sample 110. In embodiments where the third imaging objective 116 is not present, the first imaging objective 114 focuses the first beam of light towards the sample 110.

LEDs 115 illuminate the sample 110 with a second beam of light (e.g., visible light). The second beam of light can include one or more beams of light. The number of beams of light can depend on the number of LEDs 115 and/or the number of LEDs 115 that are turned on.

The first beam of light directed from the first imaging objective 114 and the second beam of light from the LEDs 115 reflect off of the sample 110. A portion of each of the first beam of light and the second beam of light returns to the imaging probe 101 via a shared imaging path.

The shared imaging path goes through the third imaging objective 116 and the first imaging objective 114 towards the second optical component 109. In some embodiments, where the third imaging objective 116 is not present, the shared imaging path goes through the first imaging objective 114 towards the second optical component 109.

The portion of the first beam of light (e.g., the first returning light) that is received by the first imaging objective 114 is directed towards the second optical component 109. The second optical component 109 reflects the first returning light towards the first optical component 107. The first optical component 107 directs the first returning light to collimator 105. The collimator 105 passes the first returning light to the waveguide 117. The waveguide 117 guides the first returning light to the OCT imaging apparatus 102.

The OCT imaging apparatus 102 senses the first returning light and provides corresponding signals to the system control and data processing unit 104. In embodiments where the third imaging objective 116 is present, the system control and data processing unit 104 can process the signals from the OCT imaging apparatus 102 to produce an OCT image of a posterior segment of the sample 110 (e.g., a retinal image of an eye). In embodiments where the third imaging objective 116 is not present, the control and data processing unit 104 processes the signals from the OCT imaging apparatus 102 to produce an OCT image of an anterior segment of the sample 110 (e.g., an image of the cornea and sclera of an eye).

The portion of the second beam of light (e.g., the second returning light) that is received by the first imaging objective 114 passes through the second optical component 109 toward reflective optical component 111. The reflective optical component 111 directs the second returning light to the second imaging objective 112. The second imaging objective 112 focuses the second returning light onto imaging camera 113.

The imaging camera 113 produces an image of the sample 110. In some embodiments, the image is a two-dimensional image of the sample 110. In embodiments where the third imaging objective 116 is present, the image can be a two-dimensional image of a posterior segment of the sample 110 (e.g., a fundus photograph of an eye). In embodiments where the third imaging objective 116 is not present, the image can be a two-dimensional image of an anterior segment of the sample 110 (e.g., an image of a cornea, sclera, iris, and/or lens of an eye).

The imaging camera 113 transmits signals indicative of the image to the system control and data processing unit 104. The system control and data processing unit 104 can synchronize the imaging capturing process between the imaging camera 113 and the OCT imaging apparatus 102. In various embodiments, the system control and data processing unit 104 synchronizes the images captured by the imaging camera 113 and the OCT imaging apparatus 102 in time and/or space.

In some embodiments, an OCT image of the anterior segment of an eye can be a three-dimensional image of a surface of the eye. In some embodiments, an OCT image (e.g., OCT volume) of an eye can be comprised of two or more cross-sectional tomographs (e.g., B-scans).

In some embodiments, the waveguide 117 is an optical fiber. In some embodiments, the waveguide 117 is used to guide the first beam of light from the OCT imaging apparatus 102 to the collimator 105 and a second waveguide (not shown) is used to guide the first returning light from the collimator 105 to the OCT imaging apparatus 102. In some embodiments, the waveguide 117 is a fiber splitter with a fixed ratio (e.g., 10/90).

In some embodiments the LEDs 115 provide light with a green wavelength (e.g., substantially 525 nm). In some embodiments, the LEDs 115 provide a light with a wavelength that is substantially different than the wavelength of light provided by the light source 103. In some embodiments, LED 115a provides light with a wavelength that is substantially different than the wavelength of the light provided by LED 115b. In some embodiments, LED 115a and LED 115b are illuminated at different times. In some embodiments, LED 115a comprises four LEDs that are continuously illuminated while imaging a sample and provide light that is substantially green on the visible light spectrum. In some embodiments, LED 115b comprises four or more LEDs that are each illuminated at different times while imaging a sample, and provide light that is substantially red or white on the visible light spectrum. In some embodiments, visible light can be provided to the sample 110 by any device known in the art that emits visible light.

In some embodiments, the first beam of light from the light source 103 has a central wavelength of approximately 830 nm and a bandwidth of approximately 65 nm.

FIG. 2 is a flow chart illustrating a method 200 for imaging a sample, according to an illustrative embodiment of the invention. The method involves illuminating a sample (e.g., an eye) with a first beam of light from a light-emitting diode (e.g., LEDs 115 as shown above in FIG. 1) and a second beam of light from an optical coherence tomography (OCT) imaging apparatus (e.g., OCT imaging apparatus 102 as shown above in FIG. 1) (Step 210).

The method 200 also involves focusing a first light reflected from the sample (e.g., second returning light as described above in FIG. 1) towards an imaging camera (e.g., imaging camera 113 as described above in FIG. 1) to obtain a first image of the sample and a second light reflected from the sample (e.g., first returning light as described above in FIG. 1) towards the OCT imaging apparatus to obtain a second image of the sample, such that the first reflected light and the second reflected light share at least a portion of an imaging path (Step 220).

FIG. 3 is a flow chart illustrating a method 300 for obtaining an image of an eye, according to an illustrative embodiment of the invention. The method involves obtaining a first optical coherence tomography (OCT) image of the eye (e.g., the OCT image obtained as described above in FIG. 1) (Step 310). In some embodiments, the OCT image size is 1024×1024 pixels. In some embodiments, the depth of the OCT image is greater than 10 mm. In some embodiments, the OCT image has depth resolution of approximately 1 μm.

The method 300 also involves obtaining a first two-dimensional image of the eye (e.g., the two-dimensional image as described above in FIG. 1) (Step 320). In some embodiments, the first two-dimensional image of the eye is captured through a telecentric flat-field imaging system mounted on a slit-lamp in a red-free mode. In some embodiments, the eye is illuminated by four low-power LEDs that emit light with a wavelength of approximately 525 nm. In some embodiments, the eye is illuminated by one or more LEDs that are located at a distance of approximately six inches from the eye.

In some embodiments, the eye is illuminated by a first set of LEDs (e.g., fixation LEDs), and a second set of LEDs (e.g., fiducial LEDs). The first set of LEDs can include four LEDs that are each illuminated at different times while imaging the eye. The second set of LEDs can include four LEDs that are continuously illuminated while imaging the eye. The first set of LEDs can each provide light with a wavelength that is substantially red on the visible light spectrum. The second set of LEDs can each provide light that is substantially green on the visible light spectrum. In some embodiments, the fixation LEDs and the fiducial LEDs provide light with any wavelength and/or combination of wavelengths on the visible light spectrum. In some embodiments, the fixation LEDs can provide light with a different wavelength on the visible light spectrum than the light provided by the fiducial LEDs. In some embodiments, the first two-dimensional image can include multiple images such that the multiple images can make a movie.

The method 300 also involves illuminating the eye (e.g., with LEDs 115 as described above in FIG. 1) at a first set of predetermined positions (Step 330). The first set of predetermined positions can be based on a set of reference positions. The reference positions can be based on a location of the eye when the first two-dimensional image of the eye is obtained. For example, if, when the first two-dimensional image of the eye was taken, the eye was gazing forward, one of the fixation LEDs can illuminate the eye such that the eye rotates to gaze up and to the right, thus illuminating the eye at first set of predetermined positions on the eye that occur when the eye rotates to gaze up and to the right. The reference positions and the first set of predetermined positions are discussed in further detail below with respect to FIGS. 5 and 6.

In some embodiments, the first set of predetermined positions are at a location a distance away from the illumination position where the first two-dimensional image was taken, such that, when the eye moves to gaze at the fixation LED for the first set of predetermined positions, the eye rotates into a position that presents to the imaging probe 101a portion of the eye that was not visible when obtaining the first two-dimensional image.

The method 300 also involves obtaining a second OCT image of the eye and a second two-dimensional image of the eye while the eye is illuminated at the first set of predetermined positions (Step 340).

The method 300 also involves determining a set of rotation reference positions based on the first two-dimensional image of the eye and the second two-dimensional image of the eye (Step 350). The set of rotation reference positions can be determined by determining a rotation matrix and/or translation matrix based on one or more positions of LED reflections shown on each of the first and second two-dimensional images of the eye. For example, in an embodiment with four LEDs illuminating the eye, the reflections of the four LEDs are visible in the first two-dimensional image of the eye and the second two-dimensional image of the eye. Setting the LED reflections in the first two-dimensional image of the eye as the reference reflections, the LED reflections of the second two-dimensional image of the eye indicate which set of predetermined positions the eye is gazing at. The predetermined positions can be used to determine the rotation matrix and/or translation matrix. The rotation matrix and/or translation matrix can be used to combine the images, as discussed in further detail below in Step 360.

In some embodiments, three or more two-dimensional images are obtained. For each two-dimensional image obtained, the eye is illuminated at a set of predetermined positions, as described above in Step 330. In some embodiments, each two-dimensional image has a set of predetermined positions that results in a different portion of the eye being imaged. For each two-dimensional image, a set of rotation reference positions can be determined based on a corresponding position of the LED reflections for each two-dimensional image of the eye and the keystone image. For example, referring to FIG. 6, a rotation matrix and/or translation matrix can be determined for images 601b, 601c, 601d, and/or 601e with respect to a location of keystone image 601a.

The method 300 also involves determining a combined image of the eye (e.g., stitched image) based on the first OCT image of the eye, the set of rotation reference positions, and the second OCT image of the eye, such that the combined image of the eye shows an area of the eye that is larger than the first OCT image of the eye and the second OCT image of the eye (Step 360). For example, as described above, the eye can be illuminated to obtain the first two-dimensional image, and then the eye can be illuminated such that the eye rotates to obtain the second two-dimensional image that shows a different portion of the eye than the first two-dimensional image. Thus, the second OCT image of the eye can include a portion of the eye that is not included in the first OCT image of the eye. The set of rotation reference positions can be used to rotate and/or translate the second OCT image of eye onto the first OCT image of the eye, to obtain a combined OCT image of the eye. For example, using the rotation and/or translation matrices described above, the second OCT image of the eye can be rotated and/or translated in a six-dimensional coordinate system having a common origin as the first OCT image of the eye. The second OCT image of the eye can be rotated and/or translated using any method currently known to those skilled in the art for rotating and translating three-dimensional images. Once the second OCT image of the eye is rotated and/or translated, the second OCT image of the eye can be superimposed onto the first OCT image of the eye, creating a combined OCT image of the eye that shows a larger portion of the eye than both the first OCT image of the eye and the second OCT image of the eye.

In some embodiments, the first set of predetermined positions is such that the first OCT image of the eye includes an entire cornea of the eye and part of a sclera. In some embodiments, the first set of predetermined positions is such that the second OCT image of the eye shows a portion of the eye that overlaps with the portion of the eye shown by the first OCT image of the eye by approximately one half.

In some embodiments, determining the combined image includes determining whether a mean height difference of the overlapping portions of the first OCT image of the eye and the second OCT image of the eye is within a desired height threshold (e.g., indicating a quality of the combination of the images). The desired height threshold can be determined by the properties of one or more lenses (e.g., imaging objective 114 and/or imaging objective 116 as described above with respect to FIG. 1) that are used to obtain the images of the eye. In some embodiments, the mean height difference threshold can be 25, 50, or 100 microns. If the mean height difference is greater than a desired height threshold, the first OCT image of the eye and the second OCT image of the eye are not combined. If the mean height difference is less than a desired height threshold, the first OCT image of the eye and the second OCT image of the eye are combined. In some embodiments, determining a mean height difference of the overlapping portions of the first OCT image of the eye and the second OCT image of the eye includes using a standard minimization procedure based on rotation and/or translation matrices of the first and second OCT images of the eye. The standard minimization procedure can begin from an initial estimate of the rotation and/or translation matrices; the initial estimate can be based on reflections of fiducial LEDs (e.g., the fiducial LEDs as described above in FIG. 3). The standard minimization procedure can be any mathematical minimization procedure currently known to those skilled in the art that can calculate a mean height value based on rotation and/or translation matrices.

In some embodiments, a first and/or second quality value is determined for the first OCT image of the eye and/or the second OCT image of the eye, respectively. Determining the first and/or second quality value can be based on a first and/or second cross-correlation between consecutive B-scans of the first OCT image and/or the second OCT image, respectively. The first cross-correlation can indicate whether consecutive B-scans for the first OCT image are sufficiently similar to use the first OCT image to combine with the second OCT image. The second cross-correlation can indicate whether consecutive B-scans for the second OCT image are sufficiently similar to use the second OCT image to combine with the first OCT image.

For example, adjacent B-scans within an OCT image of an eye (e.g., the first OCT image or the second OCT image) in which the eye did not substantially move during imaging are sufficiently similar (e.g., the location and/or color values of adjacent pixels can be approximately equal) such that the OCT image is not retaken. Adjacent B-scans within the OCT image of the eye in which the eye did substantially move (e.g., blinking, micro-saccades, and/or vergence movements) during imaging are not sufficiently similar such that the OCT image is retaken. Adjacent B-scans that are not sufficiently similar can decrease the quality of an OCT image, for example, by causing discontinuities in the OCT image in any of three dimensions (e.g., x, y, and/or z).

In some embodiments, a quality value for an OCT image of an eye can be based on a peak (e.g., magnitude) value of a cross-correlation operation performed on a pair of adjacent B-scans of the OCT image. In some embodiments, the peak value indicates that the adjacent B-scans are sufficiently similar when the peak value is above a quality threshold value (e.g., high) and not sufficiently similar when the peak value is below the quality threshold value (e.g., low). The cross-correlation operation can be any mathematical operation currently known by those skilled in the art for cross-correlating imaging data (e.g., B-scans).

The quality threshold value can be two standard deviations from a mean cross-correlation value for the OCT image. The mean-cross correlation value can be determined by determining the mean of the peak cross-correlation values where the peak cross-correlation values can be determined for every pair of adjacent B-scans in an OCT image. In some embodiments, if the OCT image has more than a desired number of pairs of adjacent B-scans whose cross-correlation value is lower than the quality threshold value, the OCT image is discarded and can be recaptured (e.g., via the imaging method 300 described with respect to FIG. 3).

FIG. 4A is a diagram 400 of an exemplary eye, according to an illustrative embodiment of the invention. More specifically, FIG. 4A shows a diagram 400 including exemplary eye 402 overlaid with five dashed rectangular shapes, each representing a transverse raster pattern of an exemplary OCT scan, a first OCT scan 401a, a second OCT scan 401b, a third OCT scan 401c, a fourth OCT scan 401d, and a fifth OCT scan 401e, generally, OCT scans 401.

FIG. 4B shows exemplary plots 410 showing OCT images acquired by the OCT scans 401 of the exemplary eye 402 from FIG. 4A, according to illustrative embodiments of the invention. More specifically, FIG. 4 shows a first OCT image 411a, a second OCT image 411b, a third OCT image 411c, a fourth OCT image 411d, and a fifth OCT image 411e, generally OCT images 411. A first OCT image 411a corresponds to a first OCT scan 401a from FIG. 4A, a second OCT image 411b corresponds to a second OCT scan 401b from FIG. 4A, a third OCT image 411c corresponds to a third OCT scan 401c from FIG. 4A, a fourth OCT image 411d corresponds to a fourth OCT scan 401d from FIG. 4A, and a fifth OCT image 411e corresponds to a fifth OCT scan 401e from FIG. 4A. OCT image 411b illustrates OCT scan 401b in a three-dimensional coordinate system after the image acquired by OCT scan 401b is rotated (e.g., by using the set of rotation reference positions obtained using the method as described above with respect to FIG. 3).

FIG. 5 is a diagram of an eye, according to an illustrative embodiment of the invention. More specifically, FIG. 5 shows a diagram 500 including a two-dimensional image of an eye captured by an imaging camera (e.g., the imaging camera 113 as described above in FIG. 1).

In some embodiments, four fiducial LEDs located in fixed positions on the imaging apparatus 100 illuminate the eye and create a fiducial pattern on the cornea of the eye indicated in FIG. 5 by a first LED reflection 501a, a second LED reflection 501b, a third LED reflection 501c, and a fourth LED reflection 501d, generally LED reflections 501. In some embodiments, a centroid of the LED fiducial pattern is aligned with the center of the pupil of the eye prior to obtaining the keystone image.

In some embodiments, four fixation LEDs are located in fixed positions on the imaging apparatus 100. In one embodiment, a position of a fixation LED that illuminates the eye to create the first LED reflection 501a can be such that when a patient gazes upon the fixation LED, the eye rotates from a reference position described by Tait-Bryan angles of R[θ, φ, ψ]=[0°, 0°, 0°], and translational coordinates (x,y,z)=(0,0,0) to a first predetermined position described by Tait-Bryan angles of R[θ, φ, ψ] [−25°, 30°, 0°], and translational coordinates (x,y,z,)=(0,0,0).

FIG. 6 shows exemplary images of a moving eye, according to an illustrative embodiment of the invention. More specifically, FIG. 6 shows a first image 601a, a second image 601b, a third image 601c, a fourth image 601d, and a fifth image 601e, generally, images 601. The images 601 each show a two-dimensional image of the eye moved into various positions.

Image 601a shows the eye in a gaze forward position and is a reference image (e.g., keystone image). Image 601b shows the eye in a gaze up and right position with respect to the gaze forward position. Image 601c shows the eye in a gaze up and left direction with respect to the gaze forward position. Image 601d shows the eye in a gaze down and right direction with respect to the gaze forward position. Image 601e shows the eye in a gaze down and left direction with respect to the gaze forward position. In various embodiments, a gaze direction other than the gaze forward position is the reference position.

A set of rotation reference positions (e.g., rotation reference positions as described above in FIG. 3) can be determined for each of the images 601b, 601c, 601d, and 601e based on the reference image 601a. The set of rotation reference positions can be determined as described above in FIG. 3. For example, for image 601b, the set of rotation reference positions is R[θ, φ, ψ]=[−25, 30, 0] and translation coordinates (x, y, z)=(0, 0, 0).

FIGS. 7A-7B show graphs 700 and 710, respectively, of images of anterior segments of an eye, according to illustrative embodiments of the invention. More specifically, FIG. 7A and FIG. 7B each show a three-dimensional image of two different porcine eyes, each created by, for example, combining OCT images as described above in FIG. 3. Each individual OCT scan (not shown) that is combined to make each of the images covers an area of approximately 16 mm×8 mm.

FIG. 8 shows a graph 800 of a mean height difference between multiple OCT images, according to an illustrative embodiment of the invention. The mean height difference between overlapping regions of each of nine exemplary OCT images with respect to a tenth exemplary OCT image is shown. The tenth exemplary OCT image height is denoted as OCT image number 3 in FIG. 8. The data point for each OCT image number in the graph of FIG. 8 shows the calculated mean height difference between that OCT image number and OCT image number 3.

While the invention has been particularly shown and described with reference to specific illustrative embodiments, it should be understood that various changes in form and detail may be made without departing from the spirit and scope of the invention.

What is claimed is:

1. An imaging probe for imaging a sample, comprising:
at least one light source that provides light to a sample;
a first imaging objective that focuses a first light reflected from the sample and a second light reflected from the sample, such that the first light and the second light share at least a portion of an imaging path; and
a first optical component that:
a) receives the first light and the second light from the first imaging objective;
b) directs the first light towards an imaging camera to obtain a first image of the sample; and
c) directs the second light toward an optical coherence tomography (OCT) imaging apparatus to obtain a second image of the sample.

2. The imaging probe of claim 1 wherein the first optical component:
directs a first beam of light received from the OCT imaging apparatus toward the first imaging objective,
the first imaging objective focuses the first bean of light toward the sample.

3. The imaging probe of claim 1 wherein the at least one light source is a light-emitting diode (LED).

4. The imaging probe of claim 1 wherein the first optical component comprises a dichroic beam splitter.

5. The imaging probe of claim 1 wherein the first optical component substantially reflects infrared light, and substantially transmits visible light.

6. The imaging probe of claim 1 further comprising a second imaging objective positioned between the imaging camera and the first optical component, the second imaging objective focuses the first beam of light toward the imaging camera.

7. The imaging probe of claim 1 further comprising a collimator positioned between the OCT imaging apparatus and the first optical component, the collimator collimates the third beam of light.

8. The imaging probe of claim 1 wherein the sample is an eye, the first image is a two-dimensional image of an anterior surface of the eye, and the second image is an OCT image of the anterior surface of the eye.

9. The imaging probe of claim 1 wherein:
the sample is an eye; and
an ophthalmic lens is positioned between the first imaging objective and the sample such that the first image is a fundus photograph of the eye, and the second image is an OCT image of the retina of the eye.

10. A method for imaging a sample, comprising:
illuminating a sample with a first beam of light from a light-emitting diode (LED) and a second beam of light from an optical coherence tomography (OCT) imaging apparatus;
focusing a reflected first light from the sample towards an imaging camera to obtain a first image of the sample and a second reflected light from the sample towards the OCT imaging apparatus to obtain a second image of the sample, such that the first reflected light and the second reflected light share at least a portion of an imaging path.

11. The method of claim 10 wherein illuminating the sample with a second beam of light further comprises
directing the second beam of light toward a first imaging objective.

12. The method of claim 10 further comprising:
illuminating the sample at a first set of predetermined positions;
obtaining a third image of the sample and a fourth image of the sample while the sample is illuminated at the first set of predetermined positions, wherein the third image of the sample is a two-dimensional image, and the fourth image of the sample is an OCT image;
determining a set of rotation reference positions based on the first image of the sample and the third image of the sample; and
determining a combined image of the sample based on the second image of the sample, the set of rotation reference positions, and the fourth image of the sample, such that the combined image of the sample shows an area of the sample that is larger than the second image of the sample and the fourth image of the sample.

13. The method of claim 10 wherein:
focusing a first reflected light further comprises focusing visible light; and
focusing a second reflected light further comprises focusing infrared light.

14. The method of claim 10 wherein focusing a first reflected light further comprises:
receiving the first reflected light by a second imaging objective; and
focusing the first reflected light toward the imaging camera.

15. The method of claim 10 wherein illuminating a sample with a second beam of light further comprises collimating the second beam of light.

16. The method of claim 10 wherein the sample is an eye, the first image is a two-dimensional image of the anterior surface of the eye, and the second image is an OCT image of the anterior surface of the eye.

17. The method of claim 10 wherein the sample is an eye, further comprising:
positioning an ophthalmic lens between the first imaging objective and the eye such that the first image is a fundus photograph of the eye, and the second image is an OCT image of the retina of the eye.

18. The method of claim 12 wherein the first set of predetermined positions is based on an amount of desired overlap between the second image of the sample and the fourth image of the sample.

19. The method of claim 18 wherein the desired overlap is half of the second image of the sample overlapping with half of the fourth image of the sample.

20. The method of claim 12 wherein determining a combined image of the sample further comprises determining whether a height difference between the second image of the sample and the fourth image of the sample is within a desired threshold.

21. The method of claim 10 further comprising determining a quality value of the second image of the sample based on an amount the sample moves during imaging.

22. A method for obtaining an image of an eye, comprising:
obtaining a first optical coherence tomography (OCT) image of the eye;
obtaining a first two-dimensional image of the eye;
illuminating the eye at a first set of predetermined positions;
obtaining a second OCT image of the eye and a second two-dimensional image of the eye while the eye is illuminated at the first set of predetermined positions;
determining a set of rotation reference positions based on the first two-dimensional image of the eye and the second two-dimensional image of the eye; and
determining a combined image of the eye based on the first OCT image of the eye, the set of rotation reference positions, and the second OCT image of the eye, such that the combined image of the eye shows an area of the eye that is larger than the first OCT image of the eye and the second OCT image of the eye.

23. The method of claim 22, further comprising:
illuminating the eye at a second set of predetermined positions, wherein the first set of predetermined positions and the second set of predetermined positions are different;
obtaining a third OCT image of the eye and a third two-dimensional image of the eye while the eye is illuminated at the second set of predetermined positions;
determining a second set of rotation reference positions based on the first two-dimensional image of the eye and the third two-dimensional image of the eye; and
determining a second combined image of the eye based on the second set of rotation reference positions, the combined image and the third OCT image of the eye, such that the second combined image shows an area of the eye that is larger than the combined image and the third OCT image of the eye.

24. The method of claim 22 wherein the first set of predetermined positions is based on an amount of desired overlap between the first OCT image of the eye and the second OCT image of the eye.

25. The method of claim 24 wherein the desired overlap is half of the first OCT image of the eye overlapping with half of the second OCT image of the eye.

26. The method of claim 22 wherein determining a combined image of the eye further comprises determining whether a height difference between the first OCT image of the eye and the second OCT image of the eye is within a desired threshold.

27. The method of claim 22 further comprising:
determining a quality value of the combination of the first OCT image of the eye with the second OCT image of the eye based on a comparison of the height difference between the first OCT image of the eye and the second OCT image of the eye with a predetermined threshold.

28. The method of claim 22, further comprising:
determining a quality value of the first OCT image of the eye based on an amount the sample moves during imaging; and
determining a quality value of the second OCT image of the eye based on an amount the sample moves during imaging.
